(19) United States
(12) Patent Application Publication
(10) Pub. No.: US 2002/0032192 A1
(43) Pub. Date: Mar. 14, 2002

(54) Treatment of vasodilatory headache

(76) Inventor: Stephen Dilly, South San Francisco, CA (US)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/784,307

(22) Filed: Feb. 16, 2001

Related U.S. Application Data
(63) Continuation of application No. PCT/GB99/02695, filed on Aug. 16, 1999.

(30) Foreign Application Priority Data
Aug. 17, 1998 (GB) 9817911.2

(51) Int. Cl. A61K 31/55; A61K 31/454; A61K 31/403

(52) U.S. Cl. 514/217.08; 514/320; 514/411

(57) ABSTRACT

A cerebral-selective anti-vasodilator e.g. of formula (I) for use in clinical conditions other than migraine resulting from undesired vasodilatation in the cerebral vasculature:

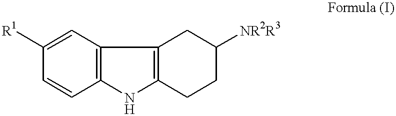

wherein:

R¹ represents hydrogen, halogen, trifluoromethyl, nitro, hydroxy, C₁₋₆alkyl, C₁₋₆—alkoxy, arylC₁₋₆alkoxy, —CO₂R⁴, —(CH₂)ₙCN, —(CH₂)ₙCONR⁵R^{6,}—(CH₂)ₙSO₂NR⁵R^{6, C}₁₋₆alkanoylamino(CH₂)ₙ, or C₁₋₆alkylsulphonyl-amino(CH₂)ₙ;

R⁴ represents hydrogen, C₁₋₆alkyl or arylC₁₋₆alkyl;

R⁵ and R⁶ each independently represent hydrogen or C₁₋₆alkyl, or R⁵ and R⁶ together with the nitrogen atom to which they are attached form a ring; n represents 0, 1 or 2; and

R² and R³ each independently represent hydrogen, C₁₋₆alkyl or benzyl or together with the nitrogen atom to which they are attached form a pyrrolidino, piperidino or hexahydroazepino ring;

and physiologically acceptable salts thereof.

This is a continuation of International Application No. PCT/GB99/02695. with an International Filing Date of Aug. 16, 1999, which claims priority to British Patent Application No. 9817911.2, filed Aug. 17, 1998, the contents of each of these applications are fully incorporated by reference.

The present invention relates to the treatment and prophylaxis of vasodilatory headaches especially those induced by nitrate drugs.

It is well established that the administration of nitrate drugs such as glyceryl trinitrate (GTN) for the treatment of angina, causes cerebral vasodilatation which induces a headache in the subject concerned. This headache may be sufficiently severe in intensity and duration that patient compliance in using the drug may be prejudicially affected. The conventional treatment of such headaches for example with paracetamol or similar analgesics is not always effective. There is therefore a need for an alternative efficacious treatment or prophylaxis of these headaches.

The effect of the anti-migraine compound sumatriptan in patients with GTN-induced headache has been described by Iversen & Olesen in Cephalalgia 16 412-418 (1996). However sumatriptan is an anti-migraine compound which causes constriction of both the cerebral an coronary vasculature and lacks any selectivity to the cerebral circulation. Also Iversen and Olesen speculate that the effect of sumatriptan in GTN-induced headache may be mediated by mechanisms shared with spontaneous migraine headaches. In fact, significant differences exist between GTN-induced headaches and migraine headaches as identified by Olesen (Trends in Pharmacological Sciences 15 149-153 (1994)). Migraine sufferers are known to be more sensitive to the effects of GTN and develop a delayed pulsating headache. GTN in normal subjects induces an immediate headache with no delayed response.

Certain carbazole derivatives have been described as anti-migraine compounds for example in published PCT patent application WO 93/00086. Of these compounds (+)-6-carboxamido-3-methylamino-1,2,3,4-tetrahydrocarbazole, in the form of its succinate salt, described in published PCT patent application WO 94/14772 has been found to be particularly effective and is currently under clinical investigation as a potential therapy for migraine under the code number VML 251 and the approved name frovatriptan. This compound differs from sumatriptan in that, at clinically relevant concentrations, it has a selective anti-vasodilatory effect against cerebral vasculature, not affecting coronary vasculature to any significant degree.

It is an object of the present invention to provide the use of a cerebral-selective anti-vasodilator such as VML 251 for the treatment or prophylaxis of clinical conditions, other than migraine, resulting from undesired vasodilatation in the cerebral vasculature.

According to one feature of the present invention therefore we provide a cerebral-selective anti-vasodilator for use in clinical conditions, other than migraine, resulting from undesired vasodilatation in the cerebral vasculature.

According to a further feature of the present invention we provide the use of a cerebral-selective anti-vasodilator in the manufacture of a medicament for use in clinical conditions, other than migraine, resulting from undesired vasodilatation in the cerebral vasculature.

According to a further feature of the present invention we provide a method for the treatment or prophylaxis of clinical conditions, other than migraine, resulting from undesired vasodilatation in the cerebral vasculature in a subject which comprises administering to the subject an effective amount of a cerebral-selective anti-vasodilator.

The above-mentioned cerebral-selective anti-vasodilators are referred to below as compounds of the invention.

As indicated above, the present invention is especially applicable to the treatment of nitrate-induced headaches especially those induced by GTN but also those induced by isosorbide mono- or di- nitrate. Another condition which may be treated in accordance with the invention is altitude sickness. Preferred anti-vasodilators for use in accordance with the invention are the carbazoles described in the above-mentioned published PCT patent application WO 93/00086 namely compounds of formula (I):

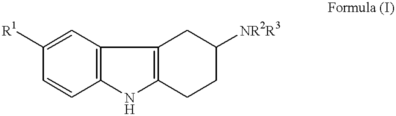

wherein:

R¹ represents hydrogen, halogen, trifluoromethyl, nitro, hydroxy, C₁₋₆alkyl, C₁₋₆-alkoxy, arylC₁₋₆alkoxy, —CO₂R⁴, —(CH₂)ₙCN, —(CH₂)ₙCONR⁵R^{6,} —(CH₂)ₙSO₂NR⁵R^{6, C}₁₋₆alkanoylamino(CH₂)ₙ, or C₁₋₆alkylsulphonyl-amino(CH₂)ₙ;

R⁴ represents hydrogen, C₁₋₆alkyl or arylC₁₋₆alkyl;

R⁵ and R⁶ each independently represent hydrogen or C₁₋₆alkyl, or R⁵ and R⁶ together with the nitrogen atom to which they are attached form a ring; n represents 0, 1 or 2; and

R² and R³ each independently represent hydrogen, C₁₋₆alkyl or benzyl or together with the nitrogen atom to which they are attached form a pyrrolidino, piperidino or hexahydroazepino ring;

and physiologically acceptable salts thereof.

Suitably R¹ represents hydrogen, halogen, cyano, hydroxy, C₁₋₆alkoxy, arylC₁₋₆-alkoxy, —CO₂R⁴, —(CH₂)ₙCONR⁵R⁶ or —(CH₂)ₙSO₂NR⁵R⁶; and R² and R³ each independently represent hydrogen or C₁₋₆alkyl.

It will be appreciated that compounds of formula (I) may contain one or more asymmetric centres, and such compounds will exist as optical isomers (enantiomers). The invention thus includes all such enantiomers and mixtures, including racemic mixtures, thereof.

In the compounds of formula (I) a halogen atom may be a fluorine, chlorine, bromine or iodine atom. An alkyl group or moiety may have a straight or branched chain. Suitable aryl groups include for example unsaturated monocyclic or bicyclic rings and partially saturated bicyclic rings of up to 12 carbon atoms, such as phenyl, naphthyl and tetrahydronaphthyl. When R⁵ and R⁶ together with the nitrogen atom form a ring, this is preferably a 5 to 7-membered saturated heterocyclic ring, which may optionally contain a further heteroatom selected from oxygen, sulphur or nitrogen. Suitable rings thus include pyrrolidino, piperidino, piperazino and morpholino.

In the above compounds R¹ preferably represents halogen (e.g. bromine), CF₃, C₁₋₆alkoxy (e.g. methoxy), (CH₂)ₙCN, —(CH₂)ₙCONR⁵R^{6,} —(CH₂)ₙSO₂NR⁵R⁶ or C₁₋₆alkanoylamino. Most preferably R¹ represents a group —(CH₂)ₙ CONR⁵R⁶ wherein n represents 0 and R⁵ and R⁶ each independently represent hydrogen, methyl, ethyl or propyl. Advantageously, R⁵ and R⁶ independently represent hydrogen or methyl.

When R¹ represents —CO₂R⁴, then R⁴ preferably represents C₁₋₆alkyl.

R² and R³ each preferably represent hydrogen, methyl or ethyl. Most preferably NR²R³ is —NH₂.

Suitable physiologically acceptable salts will be apparent to those skilled in the art and include for example acid addition salts such as those formed with inorganic acids e.g. hydrochloric, sulphuric or phosphoric acids and organic acids e.g. succinic, maleic, acetic or fumaric acid. Other non-physiologically acceptable salts e.g. oxalates may be used for example in the isolation of compounds of formula (I), and are included within the scope of this invention. Also included within the scope of the invention are solvates and hydrates of compounds of formula (I).

Examples of compounds of formula(I) include:

3-amino-6-cyano-1,2,3,4-tetrahydrocarbazole hydrochloride,

(+)-3-amino-6-carboxamido-1,2,3,4-tetrahydrocarbazole hydrochloride,

(−)-3-amino-6-carboxamido-1,2,3,4-tetrahydrocarbazole hydrochloride,

3-amino-6-methoxy-1,2,3,4-tetrahydrocarbazole hydrochloride,

3-amino-6-bromo-1,2,3,4-tetrahydrocarbazole hydrochloride,

3-amino-6-methyl-1,2,3,4-tetrahydrocarbazole oxalate,

3-amino-6-ethoxycarbonyl-1,2,3,4-tetrahydrocarbazole oxalate,

3-amino-6-(N-methyl carboxamido)-1,2,3,4-tetrahydrocarbazole hemioxalate,

3-amino-6-cyanomethyl-1,2,3,4-tetrahydrocarbazole oxalate,

3-amino-6-(N-methylsulphonamidomethyl)-1,2,3,4-tetrahydrocarbazole oxalate,

3-amino-6-chloro-1, 2,3,4-tetrahydrocarbazole oxalate,

3-amino-6-trifluoromethyl-1,2,3,4-tetrahydrocarbazole oxalate,

3-amino-6-n-butyloxy-1,2,3,4-tetrahydrocarbazole oxalate,

3-amino-6-sulphonamido-1,2,3,4-tetrahydrocarbazole oxalate,

3-amino-6-nitro-1,2,3,4-tetrahydrocarbazole oxalate,

3-amino-6-(N,N-dimethylcarboxamido)-1,2,3,4-tetrahydrocarbazole hemioxal ate,

3-amino-6-(piperidin-1-ylcarbonyl)-1,2,3,4-tetrahydrocarbazole hydrochloride,

3-amino-6-(pyrrolidin-1-ylcarbonyl)-1,2,3,4-tetrahydrocarbazole hydrochloride,

3-amino-6-(N,N-diethylcarboxamido)-1,2,3,4-tetrahydrocarbazole hydrochloride,

3-amino-6-(acetamido)-1,2,3,4-tetrahydrocarbazole oxalate,

3-amino-6-methanesulphonamido-1,2,3,4-tetrahydrocarbazole oxalate,

3-amino-6-carboxamidomethyl-1,2,3,4-tetrahydrocarbazole hydrochloride,

3-methylamino-6-carboxamido-1,2,3,4-tetrahydrocarbazole oxalate,

3-ethylamino-6-carboxamido-1,2,3,4-tetrahydrocarbazole oxalate,

3-n-propylamino-6-carboxamido-1,2,3,4-tetrahydrocarbazole oxalate,

3-i-propylamino-6-carboxamido-1,2,3,4-tetrahydrocarbazole oxalate,

3-dimethylamino-6-carboxamido-1,2,3,4-tetrahydrocarbazole oxalate,

3-benzylamino-6-carboxamido-1,2,3,4tetrahydrocarbazole oxalate,

3-pyrrolidinyl-6-carboxamido-1,2,3,4-tetrahydrocarbazole oxalate,

3-(N-(methyl)ethylamino)-6-carboxamido-1,2,3,4-tetrahydrocarbazole oxalate, and

3-amino-6-(2-carboxamidoethyl)-1,2,3,4-tetrahydrocarbazole oxalate.

As indicated above, a particularly preferred compound for use in accordance with the present invention is (+)-6-carboxamido-3-methylamino-1,2,3,4-tetrahydrocarbazole or a physiologically acceptable salt thereof especially the succinate salt including the monohydrate, which is described in published PCT patent application WO94/14772.

Compounds of formula (I) may be prepared by methods known in the art for the preparation of tetrahydrocarbazoles, for example as described in published PCT patent application WO 93/00086.

For use in medicine, the compounds of the present invention are usually administered as a standard pharmaceutical composition comprising a compound of the invention and a physiologically acceptable carrier. In a further aspect therefore the present invention provides a pharmaceutical formulation comprising a cerebral-selective anti-vasodilator such as VML 251 and a physiologically acceptable carrier, for the treatment or prophylaxis of clinical conditions, other than migraine, resulting from undesired vasodilatation in the cerebral vasculature.

The compounds of the invention may be administered by any convenient method, for example by oral, parenteral, buccal, inhalation, sublingual, nasal, rectal or transdermal administration and the pharmaceutical compositions adapted accordingly.

The compounds of the invention when given orally can be formulated as liquids, for example syrups, suspensions or emulsions, tablets, capsules and lozenges.

A liquid formulation will generally consist of a suspension or solution of the compound or physiologically acceptable salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or physiologically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochlorohydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

Preferably the composition is in unit dose form such as a tablet, capsule or ampoule.

Each dosage unit for oral administration contains preferably from 1 to 250 mg (and for parenteral administration contains preferably from 0.1 to 25 mg) of a compound of the invention.

The physiologically acceptable compounds of the invention will normally be administered in a daily dosage regimen (for an adult patient) of, for example, an oral dose of between 1 mg and 500 mg, preferably between 10 mg and 400 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the invention, the compound being administered 1 to 4 times per day. Suitably the compounds will be administered for a period of continuous therapy, for example for a week or more.

The following Examples illustrate the preparation of pharmaceutical formulations which may be employed in accordance with the present invention in which the active ingredient is a compound of formula (I) for example VML 251.

Pharmaceutical Formulations

### EXAMPLE A

A tablet for oral administration is prepared by combining

| | mg/Tablet | |
|---|---|---|
| compound of formula (I) | 100 | |
| lactose | 153 | |
| starch | 33 | |
| crospovidone | 12 | |
| microcrystalline cellulose | 30 | |
| magnesium stearate | 2 | |
| | 330 | mg |

### EXAMPLE B

An injection for parenteral administration is prepared from the following

| | | |
|---|---|---|
| compound of formula (I) | 0.50% | (w:v) |
| 1M citric acid | 30% | (v:v) |
| sodium hydroxide (qs) | to pH 3.2 | |
| water for injection BP | to 100 | ml |

The compound of formula (I) is dissolved in the citric acid and the pH slowly adjusted to pH 3.2 with the sodium hydroxide solution. The solution is then made up to 100 ml with water, sterilised by filtration and sealed into appropriately sized ampoules and vials.

1. A cerebral-selective anti-vasodilator for use in clinical conditions other than migraine resulting from undesired vasodilatation in the cerebral vasculature.

2. An anti-vasodilator according to claim 1 having the formula (I): 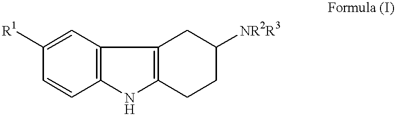
wherein:
R¹ represents hydrogen, halogen, trifluoromethyl, nitro, hydroxy, C₁₋₆alkyl, C₁₋₆-alkoxy, arylC₁₋₆alkoxy, —CO₂R⁴, —(CH₂)ₙCN, —(CH₂)ₙCONR⁵R^{6,} —(CH₂)ₙSO₂NR⁵R^{6, C}₁₋₆alkanoylamino(CH₂)ₙ, or C₁₋₆alkylsulphonyl-amino(CH₂)ₙ;
R⁴ represents hydrogen, C₁₋₆alkyl or arylC₁₋₆alkyl;
R⁵ and R⁶ each independently represent hydrogen or C₁₋₆alkyl, or R⁵ and R⁶ together with the nitrogen atom to which they are attached form a ring; n represents 0, 1 or 2; and
R² and R³ each independently represent hydrogen, C₁₋₆alkyl or benzyl or together with the nitrogen atom to which they are attached form a pyrrolidino, piperidino or hexahydroazepino ring;
or a physiologically acceptable salt thereof.

3. An anti-vasodilator according to claim 2 which is (+)-6-carboxamido-3-methylamino-1,2,3,4-tetrahydrocarbazole or a physiologically acceptable salt thereof.

4. An anti-vasodilator according to any of the preceding claims for the treatment or prophylaxis of a nitrate-induced headache.

5. The use of a cerebral-selective anti-vasodilator in the manufacture of a medicament for use in clinical conditions other than migraine resulting from undesired vasodilatation in the cerebral vasculature.

6. The use of a compound of formula (I): 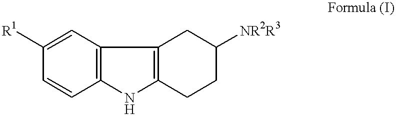
wherein R¹ R² and R³ are as defined in claim 2 or a physiologically acceptable salt thereof, in the manufacture of a medicament for use in clinical conditions other than migraine resulting from undesired vasodilatation in the cerebral vasculature.

7. Use according to claim 6 of a compound which is (+)-6-carboxamido-3-methylamino-1,2,3,4-tetrahydrocarbazole or a physiologically acceptable salt thereof

8. A method for the treatment or prophylaxis of clinical conditions, other than migraine, resulting from undesired vasodilatation in the cerebral vasculature in a subject which comprises administering to the subject an effective amount of a cerebral-selective anti-vasodilator.

9. Method according to claim 8 wherein the cerebral selective vasodilator is a compound as defined in claim 2.

10. A method for the treatment or prophylaxis of clinical conditions, other than migraine, resulting from undesired vasodilatation in the cerebral vasculature in a subject which comprises administering to the subject an effective amount of a compound which is (+)-6-carboxamido-3-methylamino-1,2,3,4-tetrahydrocarbazole or a physiologically acceptable salt thereof
